(19) United States
(12) Patent Application Publication
(10) Pub. No.: US 2010/0209371 A1
(43) Pub. Date: Aug. 19, 2010

(54) USE OF RESINS FOR STABILIZING COLORANTS

(75) Inventors: Audrey Marjorie Casado-Chaudanson, Aubagne (FR); Audrey Celine Marcelle Pastor, Antibes (FR)

Correspondence Address: YOUNG & THOMPSON, 209 Madison Street, Suite 500, Alexandria, VA, 22314, US

(73) Assignee: CHANEL PARFUMS BEAUTE, Neuilly Sur Seine (FR)

(21) Appl. No.: 12/670,748

(22) PCT Filed: Jul. 25, 2008
(86) PCT No.: PCT/FR2008/051404

§ 371 Date: Jan. 26, 2010

(30) Foreign Application Priority Data
Jul. 26, 2007 (FR) 07 05476

(51) Int. Cl. A61K 8/06; A61Q 1/02

(52) U.S. Cl. 424/63

(57) ABSTRACT

A process for preparing a cosmetic composition, characterized in that it includes: optionally a pretreatment of an organic ion-exchange resin, the absorption of at least one organic colorant onto the optionally pretreated resin in order to form a resinate, the optional grinding of the resulting resinate and the incorporation of the resinate into a cosmetically acceptable polar medium. It also relates to the composition obtained, to the resinate formed and also to the use of at least one organic ion-exchange resin for stabilizing at least one organic colorant in a cosmetically acceptable polar medium.

The present invention relates to a process for the preparation of a cosmetic composition comprising the absorption of an organic colorant on an organic ion-exchange resin optionally pretreated to form a resinate. It also relates to said resinate, the composition obtained, as well as the use of at least one organic ion-exchange resin for stabilizing at least one organic colorant in a cosmetically acceptable polar medium.

The ion-exchange resins are insoluble macromolecules bearing ionizable groups, which have the property of reversibly exchanging certain of their ions, on contact with other ions originating from a solution. These resins possess some capacity for retention of ions (expressed per gram of dry resin), which corresponds to the number of millimoles (mmol) of ions that the resin can exchange per unit of mass. The ion-exchange resins are further characterized by their particle size and by the pK of their functional group.

More particularly, the anion-exchange resins, also called anionic resins or basic resins, bear positively charged functional groups and have the property of reversibly exchanging certain of their anions, on contact with other anions such as Cl⁻, OH⁻, SO₄²⁻, etc.

The positively charged functional groups are fixed on the substrate of the resins, which can be either mineral (for example of the zeolite type) or organic (for example of the cholestyramine type, containing styrene and divinylbenzene monomer units). Among the functional groups of the anionic resins, a distinction is made between the quaternary or tertiary amine groups, which are characteristic of strong anionic resins, and the secondary and primary amine groups, which are characteristic of weak anionic resins.

Similarly, the cation-exchange resins, also called cationic resins or acid resins, bear negatively charged functional groups and have the property of reversibly exchanging certain of their cations, on contact with other cations such as Na⁺, H⁺, Ca²⁺, etc.

There are cationic resins containing styrene and divinylbenzene monomer units or acrylic monomer units, which possess sulphonic functional groups, characterizing strong cationic resins (for example Amberlite® IRP88, Amberlite® IRP69, Dowex®, etc.), or carboxylic functional groups, characterizing weak cationic resins (for example Amberlite® IRP64).

Moreover, the ion-exchange resins can be used as molecule trapping systems. The molecules thus trapped can no longer be salted-out in the non-ionic medium.

The present invention relates to this last-mentioned property of the ion-exchange resins. Among the colouring substances used in cosmetics, a distinction may be made between the mineral (or inorganic) pigments and the lakes or organic pigments.

By “lake” is meant, in the present invention, an organic colorant fixed on an inert substrate.

The organic pigments or lakes are obtained from water-soluble or liposoluble colorants by adsorption on an inert mineral substrate such as aluminium hydroxide, and rendering the colouring molecule insoluble by displacement of the sodium salt with a salt of aluminium, calcium or barium.

The organic pigments generally have high colouring power and shades that are more saturated, and therefore brighter and more attractive than those of the mineral pigments. These properties endow them with appreciable advantages, in particular for applications in make-up. However, when the lakes based on water-soluble colorants are put in an aqueous medium, a phenomenon of delaking occurs, i.e. at least partial dissolution of the colorant in the presence of water, glycols or other polar liquids, which constitute conventional components in cosmetics.

For example, owing to its irreplaceable hue, carmine (organic pigment) is very often used in the field of cosmetics, but even so it poses real problems during the manufacture of make-up products in particular because of its instability in an aqueous phase.

Now, if they are to be used in cosmetics, the organic pigments must possess good qualities of colouring and of stability, in particular during the process of manufacture of the compositions containing them.

The use of ion-exchange resins in compositions in order to permit controlled release of pharmaceutical and/or cosmetic active ingredients in and/or on the body is already known. These compositions can in addition comprise colorants.

Thus, in U.S. Pat. No. 4,788,055, dextromethorphan, an antitussive, is released in a controlled manner as a result of being trapped in a cationic resin. Colorants can optionally be added as excipients in a pharmaceutical composition.

Application US 2005/0255048 discloses a topical formulation in the form of a mousse or spray comprising a water-soluble active ingredient and a liposoluble active ingredient, with at least one of the active ingredients bound to an ion-exchange resin. The active ingredient bound to the resin is salted-out in the presence of moisture on the skin. Metal oxides can be included in the formulation.

Application US 2005/00586672 describes zeolites, mineral ion exchangers, permitting controlled release of cosmetic and/or pharmaceutical active ingredients contained in a cosmetic composition, by application of this composition, which can also include colorants, on the skin.

U.S. Pat. No. 6,033,655 describes formulations for hydrating the skin, comprising cosmetic and/or pharmaceutical active ingredients, as well as anionic or cationic resins, to which magnetic particles, thus stabilized, are bound. The formulations can comprise, apart from the resins, water-soluble colorants such as FD&C Blue 1 and/or liposoluble colorants such as D&C Green 6.

However, the use of ion-exchange resins for stabilizing organic colorants, replacing the inert substrates traditionally used in lakes, was not suggested in these documents.

Consequently, there is a need to offer systems for trapping organic colorants, enabling the phenomenon of delaking in a polar medium to be limited or even eliminated entirely.

Now, the Applicant discovered that it was possible to use, in a cosmetic composition, at least one organic ion-exchange resin for stabilizing at least one organic colorant in polar media.

It is to be understood that the invention in particular makes it possible to obtain cosmetic compositions possessing a high, stable colouring power.

The invention thus relates to a process for the preparation of a cosmetic composition, characterized in that it comprises:
a) optionally a pretreatment of an organic ion-exchange resin,
b) absorption of at least one organic colorant on the resin optionally pretreated to form a resinate,
c) optional grinding of the resinate thus obtained, and
d) incorporation of the resinate in a cosmetically acceptable polar medium.

It is to be understood that this process can comprise other preliminary, intermediate or subsequent stages, in addition to those mentioned above.

Another subject of the present invention is a cosmetic composition that can be obtained by said process.

Another subject of the invention is the cosmetic use of said composition for the care and/or make-up of keratinous materials.

Another subject of the invention is a resinate comprising at least one organic ion-exchange resin and at least one organic colorant.

Preferably, the absorption rate of an active ingredient on the resin will be 1-100% of the exchange capacity of said resin as stated in patent application US 2002/0146384.

The present invention also relates to the use of at least one organic ion-exchange resin for stabilizing at least one organic colorant in a cosmetically acceptable polar medium.

By “colorants” is meant compounds that are able to colour the skin and in particular those authorized for use as such according to the European regulations and in particular according to Directive 76/768/EEC, its annexes and its amendments.

The cosmetic colorants that can be trapped in the ionic resins according to the invention are organic colorants selected from natural colouring material of animal origin such as carminic acid (Carmine or Natural Red 4), colouring material of vegetable origin such as bixin or Natural Orange 4, norbixin, betanin; the anthocyans, the chlorophyllins, and caramel; synthetic organic colouring material such as molecules possessing at least one group selected from “nitroso” groups (Acid Green 1 etc.), “nitro” groups (Ext D&C Yellow 7 etc.), “azo” groups (Pigment Red 4, Solvent Orange 1, Solvent Red 3, Solvent Red 23, Pigment Red 57:1, Food Red 1, Acid Red 14, Acid Orange 7, FDC Yellow 6, FD&C Red 40, DC Red 33, FD&C Yellow 5 etc.), “xanthene” groups (D&C Yellow 8, D&C Orange 5, D&C Red 21, D&C Red 27, FD&C Red 3), “quinoline” groups (D&C Yellow 10 etc.), “anthraquinone” groups (Ext D&C Violet 2, DC Green 5 etc.), “indigoid” groups (FD&C Blue 2, D&C Red 30 etc.), “cyanin” groups, in particular the phthalocyanins (Pigment Blue CI-77160 etc.), “triarylmethane” groups; and mixtures thereof.

According to a preferred embodiment, the organic colorant possesses ionizable groups.

In the context of the present invention, by “resinate” is meant a complex constituted by at least one organic ion-exchange resin and at least one colorant.

By “cosmetically acceptable polar medium”, is meant a physiologically acceptable medium, i.e. compatible with the skin, which does not produce sensations of discomfort (redness, tightness, tingling etc.) that are unacceptable for the user after application on the skin, said medium containing one or more bipolar compounds capable of causing interactions and in particular bearing acid-base functions in the Lewis sense such as water, monoalcohols and/or glycols, preferably in a total amount of at least 10% and more preferably at least 20%, or even at least 30 wt. %, relative to the total weight of the composition.

The resinates according to the invention are formed by conventional techniques of absorption of ionic active ingredients on ion-exchange resins. These techniques generally comprise three main stages: an optional first stage consisting of a pretreatment of the resin, a second stage comprising the absorption proper of the colorant on the resin and a third stage comprising washing and optionally drying of the resinate obtained.

The stage of pretreatment of the resin consists of making the exchange sites of the resin available, and of removing constituents of the resin that could be exchanged subsequently. This operation can in particular be carried out by suspending the resin in various media such as water, an aqueous solution containing soda, or other solvents.

The stage of absorption of colorants comprises suspending the pretreated dry resin or the crude resin in an aqueous and/or alcoholic solution containing one or more cosmetic colorants. It is at the end of this stage that substitution between counter-ions of the resin and colorants takes place.

The washing stage consists of removing the colorant that is not bound to the resin. It is generally carried out by means of a medium in which the colorant is soluble, such as an aqueous and/or alcoholic solution.

After these three stages, a resinate is obtained. The proportion of colorant to resinate is defined as the ratio of the weight of colorant to the weight of resinate and depends entirely on the type of resin, type of colorant, conditions of treatment of the resin and conditions of absorption of the colorant on the resin.

A great many sets of conditions can be envisaged by varying certain parameters such as temperature, pressure, continuous absorption, contact time, etc. These conditions can influence the level of the colorant charge. In general, for a batch process as described in the examples (non-limitative, as they depend on the process), the level of resin in the solution for absorption can vary from 1 to 25 wt. %. The percentage of colorant in the solution for absorption is a function of its solubility. The weight ratio of colorant to resin can for example be in the range from 0.1:1 to 5:1.

For all of the conditions mentioned in the examples of absorption given below, the washing operations were carried out with a demineralized aqueous solution until the washing solutions either no longer contain free colorant or only a very low percentage.

Various washing solutions can be used. They are selected depending on the type of resin, nature of the active ingredient to be treated and of the salting-out media envisaged. Moreover, the number of washing operations required can vary considerably depending on the colorants.

In a preferred embodiment, the organic ion-exchange resin is an anionic organic resin possessing a styrene-divinylbenzene matrix such as those marketed under the trade mark Amberlite® IRA4004 Chloride by Rohm & Haas, Dowex® 2*8 Chloride by Dow Chemical Co., Dowex® 1*8 Chloride by Dow Chemical Co. and Dowex® 1*2 Chloride by Dow Chemical Co.

According to another preferred embodiment, the organic ion-exchange resin is an organic anion-exchange resin comprising acrylic monomer units such as that marketed under the trade mark Amberlite® IRA67 by Rohm & Haas.

According to an even more preferred embodiment, the organic anion-exchange resin is a resin comprising a copolymer containing styrene and divinylbenzene monomer units with quaternary ammonium functions, also called cholestyramine and in particular marketed under the trade mark Duolite® AP143/1093 by Rohm & Haas. The exchangeable anion of this resin is the chloride.

According to another preferred embodiment, the organic ion-exchange resin is an organic cationic resin comprising a copolymer containing styrene and divinylbenzene monomer units, for example the resin marketed under the trade mark Amberlite® IRP69 or a resin comprising a copolymer containing monomer units of acrylic and divinylbenzene, for example the resin marketed under the trade mark Amberlite® IRP64.

In another preferred embodiment, an anionic organic resin such as the resin Duolite® AP143/1093 is used in combination with an organic cation-exchange resin such as Amberlite® IR122 Na or Amberlite® IRP64.

Preferably, the amount of resin used in the compositions varies from 0.01 to 10 wt. %, preferably from 0.1 to 5 wt. %, relative to the total weight of the composition.

The cosmetic composition obtained according to the invention can be used for manufacturing a solid cosmetic product by a wet process, in particular for making an eye-shadow or a blusher. For example, it can be employed in a particular process of manufacture of a baked make-up, comprising the stages consisting of:
1) introducing, in a mould provided with a bottom and an opening, a composition according to the invention containing at least one resinate and at least one fluid binder comprising a volatile solvent;
2) placing on said composition, in direct contact with it, a plate of porous material having an average pore diameter in the range from 5 to 180 μm;
3) passing at least a portion of the fluid binder through said plate;
4) removing the fluid binder that has passed through said plate to obtain a partially solidified composition;
5) drying said partially solidified composition to obtain a solidified composition;
6) optionally withdrawing said mould and/or said plate; and
7) placing said solidified composition in packaging, in such a way that the surface previously in contact with the bottom of the mould is visible.

In these methods, by avoiding delaking and thus rising of the colorant to the surface, the final product does not lose its colour.

As a variant, the cosmetic composition according to the invention can be used in a process of granulation by a wet process. The colorant retained in the resin does not leave in the aqueous phase during the granulation stage and thus the final product has a correct colour.

Another advantage of the invention arises from the fact that by avoiding delaking of the colorant, the latter, once applied on the skin, does not become fixed on the deep-lying keratin and therefore is easier to remove from the skin during make-up removal.

The composition according to the invention can be in any pharmaceutical form and in particular in the form of powder, gel, dispersion or oil-in-water (O/W), water-in-oil (W/O) or multiple (W/O/W, O/W/O, etc.) emulsion.

The composition according to the invention can contain various additives, such as at least one compound selected from:
oils, which can in particular be selected from: linear or cyclic, volatile or non-volatile silicone oils, such as polydimethylsiloxanes (dimethicones), polyalkylcyclosiloxanes (cyclomethicones) and polyalkylphenylsiloxanes (phenyldimethicones); synthetic oils such as fluorinated oils, alkyl benzoates and branched hydrocarbons such as polyisobutylene; vegetable oils and in particular soya oil or jojoba oil; and mineral oils such as paraffin oil;
waxes, such as ozokerite, polyethylene wax, beeswax or carnauba wax;
silicone elastomers obtained in particular by reaction, in the presence of a catalyst, of a polysiloxane having at least one reactive group (in particular hydrogen or vinyl) and bearing at least one alkyl (in particular methyl) or phenyl, end and/or side group, with an organosilicone such as an organohydrogenpolysiloxane;
surfactants, preferably emulsifiers, whether non-ionic, anionic, cationic or amphoteric, and in particular esters of fatty acids and of polyols such as esters of fatty acids and glycerol, esters of fatty acids and sorbitan, esters of fatty acids and polyethylene glycol and esters of fatty acids and sucrose; ethers of fatty alcohols and of polyethylene glycol; alkylpolyglucosides; and modified polyether polysiloxanes; betaine and its derivatives; polyquaterniums; ethoxylated fatty alcohol sulphate salts; sulphosuccinates; sarcosinates; alkyl- and dialkyl phosphates and their salts such as potassium cetyl phosphate; and soaps of fatty acids;
co-surfactants such as linear fatty alcohols and in particular cetyl and stearyl alcohols;
thickeners and/or gelling agents, and in particular crosslinked or non-crosslinked, hydrophilic or amphiphilic homopolymers and copolymers, of acryloylmethylpropane sulphonic acid (AMPS) and/or of acrylamide and/or of acrylic acid and/or of acrylic acid salts or esters; xanthan gum or guar gum; cellulose derivatives; and silicone gums (dimethiconol);
humectants, such as polyols, including glycerol, propylene glycol and sugars, and glycosaminoglycans such as hyaluronic acid and its salts and esters;
organic sunscreens, such as derivatives of dibenzoylmethane (including butyl methoxydibenzoylmethane), esters of cinnamic acid (including ethylhexyl methoxycinnamate), salicylates, β-β′-diphenylacrylates, triazines, and phenylbenzotriazoles;
inorganic sunscreens, based on mineral oxides in the form of pigments or nanopigments, coated or uncoated, and in particular based on titanium dioxide or zinc oxide,
preservatives;
compounds with an optical effect such as fillers, pigments, nacres or interference pigments, lifting agents, mattifying polymers and mixtures thereof;
sequestering agents such as EDTA salts;
perfumes;
and mixtures thereof, this list not being limitative.

By “fillers” is meant colourless or white, mineral or synthetic, lamellar or non-lamellar particles, intended to give body or stiffness to the composition and/or softness, mat appearance and immediate uniformity on application. As fillers, there can in particular be mentioned talc, mica, silica, kaolin, nylon powders such as of Nylon-12, for example marketed under the trade mark Orgasol® by the company Atochem, polyethylene powders, polyurethane powders, polystyrene powders, polyester powders, optionally modified starch, microbeads of silicone resin, such as for example those marketed by the company Toshiba under the name Tospearl®, hydroxyapatite, and hollow silica microspheres, for example marketed under the trade mark Silica Beads® by the company Maprecos.

By “pigments” is meant white or coloured, mineral or organic particles, insoluble in the medium, intended to colour and/or opacify the composition, of ordinary or nanometric size. Among the mineral pigments, there can be mentioned the dioxides of titanium, of zirconium or of cerium, as well as the oxides of zinc, of iron or of chromium.

By “nacres or interference pigments” is meant light-reflecting iridescent particles. Among the nacres that may be considered, there can be mentioned natural nacre, mica covered with titanium oxide, with iron oxide, with natural pigment or with bismuth oxychloride as well as coloured titanium mica.

By “lifting agent” is meant compounds that can have a pulling effect, i.e. which can stretch the skin and, by this stretching effect, can smooth the skin and reduce or even immediately eliminate wrinkles and lines. As lifting agents, there can be mentioned polymers of natural origin.

By “polymer of natural origin” is meant polymers of vegetable origin, polymers derived from the integumentary appendages, proteins from egg and latexes of natural origin. These polymers are preferably hydrophilic. As polymers of vegetable origin, there can in particular be mentioned proteins and hydrolysates of proteins, and more particularly extracts of cereals, of leguminous plants and of oleaginous plants, such as extracts of maize, of rye, of wheat, of buckwheat, of sesame, of spelt, of pea, of bean, of lentil, of soya and of lupin. The synthetic polymers in the form of a latex or a pseudolatex can be of the polycondensate type or of the radical type. There can in particular be mentioned the copolymer of PVP/dimethiconylacrylate and of hydrophilic polyurethane marketed for example under the trade mark Aquamere® S-2011 by the company Hydromer.

By “mattifying polymers” is meant, in the present invention, any polymer in solution, in dispersion or in the form of particles, which prevents the skin from being shiny and which makes the complexion uniform. There can be mentioned, for example, silicone elastomers, resin particles, and mixtures thereof. As silicone elastomers, there can be mentioned, for example, the products marketed under the names “KSG®” by the company Shin-Etsu, under the names “Trefil®”, “BY29®” or “EPSX®” by the company Dow Corning or under the names “Gransil®” by the company Grant Industries.

The concentration of these fillers and/or pigments and/or nacres by weight in the aqueous phase is generally from 0.1 to 20 wt. %, and preferably from 0.2 to 7 wt. % relative to the total weight of the composition.

Examples of said additives are mentioned in particular in the CTFA Dictionary (International Cosmetic Ingredient Dictionary and Handbook published by The Cosmetic, Toiletry and Fragrance Association, 11th Edition, 2006).

The composition used according to the invention can moreover comprise cosmetic active ingredients and in particular at least one active ingredient selected from: agents that stimulate the production of growth factors; anti-glycation or deglycating agents; agents that increase the synthesis of collagen or prevent its degradation (anti-collagenase agents, in particular inhibitors of matrix metalloproteinases); agents that increase the synthesis of elastin or prevent its degradation (anti-elastase agents); agents that increase the synthesis of glycosaminoglycans or of proteoglycans or prevent their degradation; agents that increase the proliferation or differentiation of keratinocytes; agents that increase the proliferation of fibroblasts; depigmenting, anti-pigmenting or propigmenting agents; antioxidant or anti-radical or anti-pollution agents; agents that increase the synthesis of epidermal lipids; agents that stimulate lipolysis, inhibit lipogenesis and/or inhibit the differentiation of adipocytes; agents that drain or detoxify or improve the microcirculation; and mixtures thereof, without this list being limitative.

Examples of these additional active ingredients are in particular: plant extracts and in particular the extracts of Chondrus crispus, Thermus thermophilus, Pisum sativum, Centella asiatica, Sphacelaria scoparia, Scenedesmus, Moringa pterygosperma, ivy (Hedera helix), Castanea sativa, Hibiscus sabdriffa, Polyanthes tuberosa, Argania spinosa, seeds of Hibiscus esculentus, Narcissus tarzetta, liquorice or Ruscus esculatus; an essential oil of Citrus aurantium (Neroli); silicon derivatives such as methylsilanol mannuronate; vegetable protein hydrolysates (in particular of soya or hazelnut); sugars; acylated oligopeptides, in particular marketed by the company SEDERMA under the trade names Maxilip®, Matrixyl® 3000, Biopeptide® CL or Biopeptide® EL or described in application EP-1 449 517; yeast extracts and in particular of Saccharomyces cerevisiae; extracts of algae and in particular of Laminaria and of Blidingia minima; caffeine and extracts of coffee and of maté containing it; vitamins and their derivatives such as retinyl palmitate, ascorbyl glucoside, ascorbyl palmitate, ascorbyl tetraisopalmitate, ascorbyl sorbate, tocopherol, tocopheryl acetate and tocopheryl sorbate; homopolymers and copolymers of methacryloyloxyethylphosphorylcholine; urea; ceramides and phospholipids; arbutin; dihydroxyacetone (DHA), erythrulose; and mixtures thereof.

The composition can also contain at least one colouring material selected from water-soluble or liposoluble colorants, fillers having the effect of colouring and/or opacifying the composition and/or of colouring the lips, such as pigments, nacres, lakes, and mixtures thereof. This colouring material can optionally be surface-treated with a hydrophobic agent such as silanes, silicones, soaps of fatty acids, C₉₋₁₅ fluoroalcohol phosphates, acrylate/dimethicone copolymers, C₉₋₁₅ fluoroalcohol phosphate/silicone mixed copolymers, lecithins, carnauba wax, polyethylene, chitosan and amino acids, optionally acylated, such as lauroyl lysine, disodium stearoyl glutamate and aluminium acyl glutamate. The pigments can be mineral or organic, natural or synthetic. Examples of pigments are in particular oxides of iron, of titanium or of zinc, as well as composite pigments and goniochromatic, pearlescent, interference, photochromic or thermochromic pigments, without this list being limitative. The nacres can be selected from those conventionally present in make-up products, such as titanium micas.

The composition according to the invention can be in the form of a pencil, mascara, eye liner, foundation, lip gloss, lipstick, lip-care stick, body make-up product, eye-shadow or blusher, concealer, or care product for the face or the body.

The invention will now be illustrated with the following non-limitative examples.

### EXAMPLES

### A—Tests In Vitro

### Example 1

### Examples of the Pretreatment of Anionic Resins

### Condition 1.1:

20 g of resin Duolite® AP143/1093 (supplier: Rohm & Haas) is suspended in 200 g of water, it is stirred and is then left to settle, and the supernatant is removed. The operation of washing with demineralized water is repeated about 10 times. Then the same operation is carried out with a 10% NaCl solution (also about 10 times), followed by rinsing with demineralized water. Then the wet resin is dried in a ventilated stove at 35° C. overnight.

### Condition 1.2:

20 g of resin Duolite® AP143/1093 (supplier: Rohm & Haas) is suspended in 200 g of water with 10% NaOH, it is stirred and then left to settle, and the supernatant is removed (the operation is repeated about 10 times). Then it is rinsed twice with demineralized water and the wet resin is dried in a ventilated stove at 35° C. overnight.

### Example 2

### Examples of the Absorption of Colorants on Different Resins

### Condition 2.1: the Solution for Absorption of the Colorant Contains 0.5% of Methylene Blue, a Cationic Dye

About 1.2 g of crude resin Amberlite® IRP64 is suspended in 60 g of aqueous solution containing 0.5% of Methylene Blue. It is stirred for about 1 hour, washed with demineralized water, the supernatant is removed and the resin charged with Methylene Blue is dried. The charge of Methylene Blue on the resin was not evaluated.

### Condition 2.2: the Solution for Absorption of the Colorant Contains 0.5% of Water-Soluble Carmine, an Anionic Colorant

About 1.2 g of crude resin Duolite® AP143/1093 is suspended in 60 g of aqueous solution containing 0.5% of water-soluble carmine or carminic acid. It is stirred for about 1 hour, washed with demineralized water, the supernatant is removed and the resin charged with water-soluble carmine is dried. The charge of water-soluble carmine on the resin was not evaluated.

### Condition 2.3: the Solution for Absorption of the Colorant Contains 5% of Water-Soluble Carmine, an Anionic Colorant

About 1.2 g of crude resin Duolite® AP143/1093 is suspended in 60 g of aqueous solution containing 5% of water-soluble carmine or carminic acid. It is stirred for about 1 hour, washed with demineralized water, the supernatant is removed and the resin charged with water-soluble carmine is dried. The charge of water-soluble carmine on the resin was not evaluated.

### Example 3

### Tests on Resinates of Colorants

### Condition 3.1: Influence of the Absorption Conditions

On comparing the colour of the resinates of water-soluble carmine obtained for conditions 2.2 and 2.3, it is found that the colour depends on the level of water-soluble carmine present in the absorption solution.

### Condition 3.2: Aqueous Phase Stability

Resin that has been subjected to the absorption conditions 2.1 is suspended in an aqueous phase. This stability test therefore relates to resinates of Methylene Blue.

The colour of the medium is evaluated using a Minolta-CT-310 camera.

For this, the Lab parameters of the sample are measured. The Lab colour space (also called CIELAB space) is one of the uniform colour spaces defined by the CIE in 1976. The parameter L is a lightness variable whereas the parameters a and b are chromaticity coordinates.

The following values are obtained: L=99.55; a=−0.67; b=0.05

Then a colour difference (also called difference in shade) ΔE* is evaluated by comparing this sample with a neutral aqueous phase whose Lab parameters are: L=100.01; a=−0.05; b=0.05.

The colour difference ΔE* in the Lab colour space indicates the degree of colour difference but not the direction, it is defined according to the following equation:

ΔE*={square root over (((L₁−L₂)²+(a₁−a₂)²+(b₁−b₂)²)}

When ΔE*<2, the two samples are not significantly different, and vice versa.

ΔE*=0.8 is obtained

The medium containing the resinate of Methylene Blue is very similar to a neutral aqueous phase (according to the Lab measurements and the value of ΔE*), which confirms that in this sample, there was no release of Methylene Blue in the water.

Conditions 3.3: Comparison with an Unstabilized Colorant

The objective of these tests is to compare the aqueous phase stability of resinates of water-soluble carmine with two samples of carmine commonly used in cosmetics: one in free, water-soluble form and the other fixed on an inert substrate, insoluble in water and oil.

The products tested are as follows:
Free, water-soluble carmine: carminic acid, aluminium-calcium salt
Carmine fixed on an inert substrate (Covalac® carmine): Aluminium lake of carmine or carminic acid
Water-soluble carmine resinate prepared according to conditions 2.2
Water-soluble carmine resinate prepared according to conditions 2.3

The process comprises the following stages:
Each product is suspended in aqueous solution (0.007 g/10 g of aqueous phase),
A vortex is created for about 1 min,
They are left in contact for about 5 hours,
The supernatant is filtered, and
The Lab parameters of each sample are measured.

The colour difference ΔE* of each sample is then evaluated by comparison with a neutral aqueous phase the Lab parameters of which are: L=100.01; a=−0.04; b=0.04.

| | Free, water- | | Resinate | Resinate |
|---|---|---|---|---|
| | soluble carmine = | Covalac ® | (condition | (condition |
| | control | carmine | 2.2) | 2.3) |
| ΔE* | 115.0 | 59.3 | 0.3 | 0.1 |

For the resinates, ΔE* is much lower than 2, indicating that there is no difference between the aqueous phases containing them and the neutral aqueous phase.

For the carmine fixed on an inert substrate (Covalac® Carmine) and the free, water-soluble carmine, ΔE* is far higher than 2, indicating that the aqueous phase is very different from a neutral aqueous phase.

These tests thus show that the delaking of colorant is very marked with the carmine fixed on an inert substrate such as aluminium (Covalac® Carmine) whereas delaking is completely absent when resinates of water-soluble carmine are used.

Conditions 3.4: Comparison with an Encapsulated Colorant

The products tested are as follows:
Encapsulated carmine Test 1A: Encapsulation of Covalac® Carmine in waxes
Encapsulated carmine Test 2: Encapsulation of Covalac® Carmine in waxes
Water-soluble carmine resinate prepared according to conditions 2.2
Water-soluble carmine resinate prepared according to conditions 2.3

The process comprises the following stages:
Each product is suspended in aqueous solution (0.007 g/10 g of aqueous phase),
A vortex is created for 1 min,
They are left in contact for 3 months at 45° C.,
The supernatant is filtered, and
The Lab parameters of each sample are measured.

Then the colour difference ΔE* of each sample is evaluated by comparison with a neutral aqueous phase whose Lab parameters are: L=100.01; a=−0.05; b=0.05.

| | Encapsulated | Encapsulated | Resinate | Resinate |
|---|---|---|---|---|
| | carmine | carmine | (condition | (condition |
| | Test 1A | Test 2 | 2.2) | 2.3) |
| ΔE* | 73.9 | 7.5 | 0.1 | 0.1 |
| after 1 | | | | |
| month | | | | |
| ΔE* | 83.6 | 19.7 | 0.1 | 0.1 |
| after 2 | | | | |
| months | | | | |
| ΔE* | 86.5 | 32.5 | 0.2 | 0.1 |
| after 3 | | | | |
| months | | | | |

It can be seen that only the samples containing a resinate have a ΔE* less than 2, which means that only these samples have not undergone delaking of colorant, even after three months.

These results show that the resinates of water-soluble carmine make it possible to prevent the delaking of carmine in aqueous phase, which is not made possible by encapsulation of the carmine.

### B—Cosmetic Composition

A composition containing the following ingredients identified (in upper case) by their INCI names or (in lower case) by their function, the amounts being stated as percentage by weight, was prepared in a manner known by a person skilled in the art.

The resin used is the one that was subjected to absorption conditions 2.2.

### Example

Pulverulent phase:

| | |
|---|---|
| MICA & BISMUTH OXYCHLORIDE | 7.60% |
| Magnesium stearate | 1.20% |
| MICA | 14.68% |
| Preservatives | 0.32% |

Resinate:

| | |
|---|---|
| carmine 0.5%/Duolite ® AP143/1093 | 3.20% |
| Pigments (iron oxides) | 9.40% |
| MANGANESE VIOLET | 3.60% |

Binder phase:

| | |
|---|---|
| WATER | 56.07% |
| DIMETHICONE | 0.30% |
| MAGNESIUM ALUMINIUM SILICATE | 0.90% |
| BUTYLENE GLYCOL | 0.60% |
| POLYSORBATE 20 | 0.90% |
| SORBITOL & WATER | 1.20% |
| Preservative | 0.03% |

A pasty composition is obtained, which can be used for the manufacture of make-up.

1-13. (canceled)

14. Process for the preparation of a cosmetic composition in the form of a powder, a gel or an oil-in-water, a water-in-oil or a multiple emulsion, which comprises:
absorption of at least one organic colorant on an organic ion-exchange resin to form a resinate,
incorporation of the resinate in a cosmetically acceptable polar medium.

15. Process according to claim 14, which further comprises the preliminary step of pretreating the resin.

16. Process according to claim 14, which further comprises the step of grinding the resinate before incorporation in the polar medium.

17. Process according to claim 14, wherein the organic colorant is selected from natural colouring material of animal origin, colouring material of vegetable origin; synthetic organic colouring material; and mixtures thereof.

18. Process according to claim 14, wherein the colorant is water-soluble.

19. Process according to claim 14, wherein the organic ion-exchange resin is an organic anion-exchange resin.

20. Process according to claim 19, wherein the organic anion-exchange resin comprises a copolymer containing acrylic and divinylbenzene monomer units.

21. Process according to claim 19, wherein the organic anion-exchange resin comprises a copolymer containing styrene and divinylbenzene monomer units.

22. Process according to claim 14, wherein the organic ion-exchange resin is an organic cation-exchange resin.

23. Process according to claim 22, wherein the organic cation-exchange resin comprises a copolymer containing styrene and divinylbenzene monomer units.

24. Process according to claim 22, wherein the organic cation-exchange resin comprises a copolymer containing acrylic and divinylbenzene monomer units.

25. Process according to claim 14, wherein a combination of organic anion-exchange resin and organic cation-exchange resin is used.

26. Cosmetic composition obtained by the process according to claim 14.

27. A cosmetic process for the care and/or make-up of keratinous materials, comprising applying onto skin a composition according to claim 14.

28. Resinate obtained by pretreatment of an organic ion-exchange resin and absorption of at least one organic colorant on the resin.

29. Process for stabilizing at least one organic colorant in a cosmetically acceptable polar medium, comprising absorbing said colorant on at least one organic ion-exchange resin.
